(19) United States
(12) Patent Application Publication
(10) Pub. No.: US 2024/0156396 A1
(43) Pub. Date: May 16, 2024

(54) IMPLANTABLE SENSOR FOR DETERMINING ORIENTATION AND MOVEMENT OF BONE

(71) Applicant: ORTHOSOFT ULC, Montreal (CA)

(72) Inventors: Louis-Philippe AMIOT, Montreal (CA); Joseph MADIER VIGNEUX, Montreal (CA); Sharif SHARIFZADEH, Montreal (CA); Karine DUVAL, Montreal (CA)

(21) Appl. No.: 18/508,668

(22) Filed: Nov. 14, 2023

Related U.S. Application Data
(60) Provisional application No. 63/424,998, filed on Nov. 14, 2022.

(51) Int. Cl. A61B 5/00

(52) U.S. Cl. CPC A61B 5/4504; A61B 5/0031; A61B 5/6878; A61B 5/6882; A61B 5/002; A61B 2562/028

(57) ABSTRACT

An implantable sensing device may include a housing. An implantable sensing device may include onboard electronics including one or more sensors carried by the housing. An implantable sensing device may include one or more anchoring features coupled to the housing and extending outward thereof, wherein the one or more anchoring features are configured to engage with the bone at a medullary canal thereof to couple the implantable device with the bone.

### CROSS-REFERENCE TO RELATED APPLICATION

The present application claims the priority of U.S. Patent Application No. 63/424,998, filed on Nov. 14, 2022 and incorporated herein by reference.

### TECHNICAL FIELD

The present disclosure is directed to systems, devices and methods incorporating sensors for use in performing, monitoring and evaluating medical procedures, such as arthroplasty procedures.

### BACKGROUND

Arthroplasty procedures involve the use of specialized tools and the implantation of medical devices such as orthopedic implants, into a patient. These orthopedic implants can replicate one or more portions of a joint from which bone has been removed. Typically, once the orthopedic implant is implanted into the patient, or even while it is being implanted, it is difficult to obtain feedback regarding the effectiveness of the implant or the implant procedure. Attempts have been made to obtain data from orthopedic implants using sensors. Efforts in this area are still being actively pursued and refined. However, such “smart” orthopedic implants can be costly, may require redesigns, and can suffer from incomplete sensor data, battery life limitations, and infrequent data collection.

Computer-assisted surgery (CAS) systems such as those that employ inertial-based or microelectro-mechanical sensor (MEMS), trackable members have been developed. One of the principal steps in navigating a bone with inertial sensors is to determine a coordinate system of the bone relative to the sensors, so as to be able to track the orientation of the bone. However, bone axis digitizer devices that support MEMS typically must have multi-point attachments to eliminate movement, may be bulky, and can experience accidental displacement during surgery.

### Overview

The present subject matter can provide a solution to these and other problems, such as by providing a dedicated smart implant with sensing capability. This smart implant can be utilized with various tools as part of the CAS system and can be used in various bones of the body by being configured for implantation into a medullary canal of the bone, for example. Placement in the medullary canal can be advantageous in determining an orientation of a mechanical axis of the bone as the medullary canal is arranged along the mechanical axis and determining bone movement, among other benefits. More particularly, placing the smart implant in the medullary canal can reduce or eliminate drawbacks experienced with typical MEMS as such placement in the medullary canal can reduce the chances of accidental displacement during surgery.

Providing for a separate smart implant from surgical tool(s) and/or orthopedic implant(s) can allow for use of such tool(s) and/or orthopedic implants without requiring a costly redesign to make such components smart. The smart implant can be configured to be coupled to the anatomy and further can be coupled to one or more of the tool(s) to provide a reference from which the tool(s) can be oriented. The smart implant can be fabricated in a universally applicable design that can be adapted for use in different anatomies.

The smart implant can be insertable and removable from the medullary canal of the bone as desired. The present inventors recognize that the smart implant(s) disclosed herein may be temporary components utilized during trialing. The smart implant can then be removed from the anatomy after or during trialing, which can reduce or alleviate concerns about complete sensor data, short battery life, infrequent data collection and other deficiencies. Thus, one contemplated use of the smart implant is during trialing. During a surgical arthroplasty procedure to implant a prosthetic knee joint, trialing involves performing range of motion and other determinations, use of tools such as cut guides to remove diseased bone from the joint and the use of one or more provisional components to obtain proper sizing for permanent orthopedic implants.

### BRIEF DESCRIPTION OF THE DRAWINGS

FIG. 1 is a schematic view of a sensor system including a smart implant that is independently implantable into anatomy of a patient for use in computer-assisted surgery.

FIG. 2A is a perspective view of the smart implant of FIG. 1.

FIGS. 2B and 2C are plan views of the smart implant of FIGS. 1 and 2A.

FIG. 3 is a schematic view of the smart implant of FIGS. 1-2C inserted into a medullary canal of a long bone according to a first method.

FIG. 4 is a schematic representation of a second method of anchoring a second example of the smart implant temporarily within a medullary canal of a second long bone.

FIG. 5 is a schematic representation of a third method of anchoring a third example of the smart implant temporarily within the medullary canal of the second long bone.

FIG. 6 is a schematic representation of a fourth method of anchoring a fourth example of a smart implant within the medullary canal of the second long bone with an insertion tool extending to a location external of the long bone.

FIG. 7 is a schematic representation of a fifth method of anchoring a fifth example of the smart implant within the medullary canal of the second long bone with an anchoring device extending to a location external of the long bone.

FIG. 8 is a schematic view of various electronic and mechanical components of an exemplary smart implant.

FIG. 9 is a flow diagram of a method of monitoring one or more characteristics of a patient using a sensing device according to an example of the present application.

### DETAILED DESCRIPTION

CAS has been developed in order to help a surgeon to alter bones, and to position or orient implants to a desired location. CAS may encompass a wide range of devices, including surgical navigation, pre-operative planning, trialing and various robotic devices. Many conventional techniques of joint arthroplasties do not use a robot, which can result in errors or can lack precision. CAS systems can help to reduce errors and increase precision. CAS can be improved by making a better determination of a location/orientation of bone(s) and instruments as it relates to the bone(s). This improvement can help improve accuracy of positioning for cutting operations performed, in part or in whole, by the CAS system. However, existing tracking devices of CAS can be improved. This disclosure provides for improvements with respect to bone tracking and other sensing of patient characteristics.

The smart implants, methods and systems described herein can be used as part of a CAS system such as an inertial-based CAS system employing trackable members having inertial-based sensors. The inertial-based CAS system can utilize sensors such as the micro-electro-mechanical sensors (MEMS) based system and methods disclosed in co-pending U.S. Provisional Patent Applications, entitled SYSTEMS, METHODS, AND APPARATUSES FOR TIBIAL MECHANICAL AXIS DIGITIZATION and COMPUTER-ASSISTED TIBIA RESECTION filed on the even day with the present case and disclosed in U.S. Pat. Nos. 10,874,405, 10,729,452, 9,901,405, 9,839,533 and 8,265,790, the entire contents of each of which are incorporated herein in its entirety by reference. However, it is to be understood that the smart implants, methods and systems described herein may also be used with other computer-assisted surgery (CAS) systems such as those using Rosa® Robotic Technology and/or with other tracking modalities, such as optical tracking. It is further contemplated that the smart implants, although described herein as temporary implants used during trialing, could be utilized as permanent smart implants to provide postoperative sensing capability after implantation of traditional orthopedic implants. The term “bone” as used herein is not limited to the tibia but can include any applicable bone of the body including the humerus, femur, etc. Although the examples are described herein in reference to mounting of the smart implant in the tibial or femoral medullary canal and reference a knee arthroplasty, the apparatuses, systems, techniques and methods discussed herein are not so limited and can be used in other anatomic locations such as adjacent other joints such as the spine, shoulder, hip, ankle, wrist or the like.

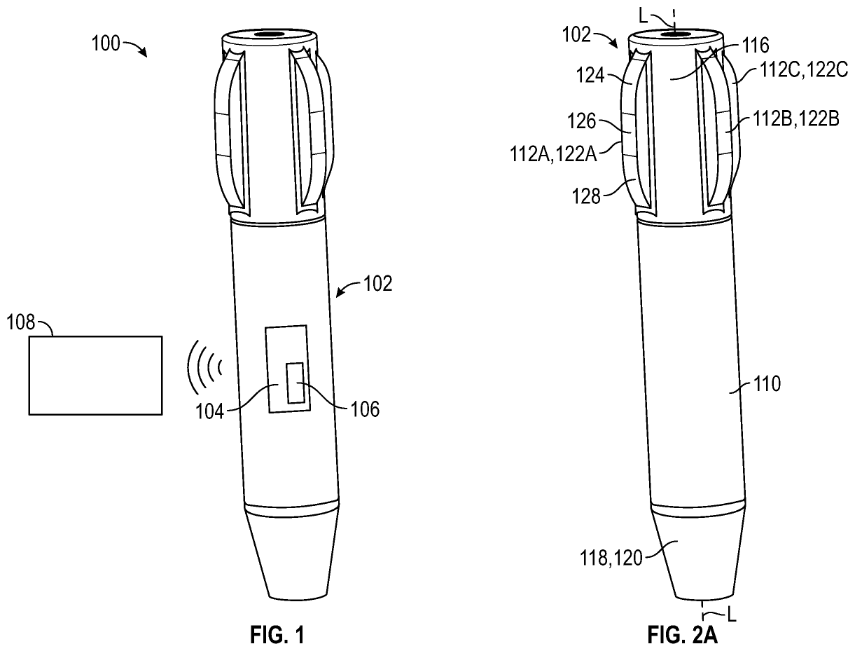

FIG. 1 shows a schematic example of a sensor system 100 according to an example. The sensor system 100 can include a smart implant 102 having onboard electronics 104 including one or more sensors 106 and one or more electronic device(s) external of the patient. These one or more electronic device(s) are referenced herein as a controller 108 for simplicity.

The onboard electronics 104 of the smart implant 102 including the one or more sensors 106 will be described in further detail subsequently. Thus, only a brief overview is provided. The one or more sensors 106 can be configured to collect data on one or more patient characteristics including but not limited to orientation of the smart implant 102 as dictated by the orientation of the bone, movement of the smart implant 102 as dictated by movement of the bone, temperature, pH, etc. Thus, the one or more sensors 106 can include any one or combination of different types of sensors (e.g., an accelerometer, a gyroscope, a compass, an electronic tilt sensor, a piezoelectric sensor, force sensor, thermometer, pH monitor, strain gauge, or any combination or multiples thereof, including any other sensor that can be used to detect motion within a body). This data can be transmitted wirelessly (or via a wired connection) to the controller 108.

The controller 108 can include one or more processors, microprocessors, microcontrollers, electronic control modules (ECMs), electronic control units (ECUs), programmable logic controller (PLC), or any other suitable means for electronically communicating with the smart implant 102. The controller 108 can be configured to operate according to a predetermined algorithm or set of instructions for communicating with smart implant 102. Such an algorithm or set of instructions can be stored in a database, can be read into an on-board memory of the controller 108, or preprogrammed onto a storage medium or memory accessible by the controller 108, for example, in the form of a floppy disk, hard drive, optical medium, random access memory (RAM), read-only memory (ROM), or any other suitable computer-readable storage medium commonly used in the art (each referred to as a “database”), which can be in the form of a physical, non-transitory storage medium.

The controller 108 can be in electrical communication or connected to a display (not shown), or the like, and various other components, or multiple smart implants, like smart implant 102. By way of such connection, the controller 108 can receive data pertaining to bone orientation and/or bone movement as captured by the smart implant 102. In response to such input, the controller 108 can perform various determinations and transmit output signals corresponding to the results of such determinations or corresponding to actions that need to be performed, such as alerting the surgeon, making recommendation to the surgeon, robotically and without surgeon guidance, implementing orienting one or more cut guides as appropriate based upon the sensed bone orientation and/or bone movement as captured by the smart implant 102, etc.

The controller 108, including a human-machine interface, can include various output devices, such as screens, video displays, monitors and the like that can be used to display information, warnings, data, such as text, numbers, graphics, icons, and the like, regarding the status or data captured by smart implant 102. The controller 108, including the human-machine interface, can additionally include a plurality of input interfaces for receiving information and command signals from various sensors associated with the CAS system, the smart implant 102 and/or other surgical tools and a plurality of output interfaces for sending control signals to various components of the CAS system including the smart implant 102. Suitably programmed, the controller 108 can serve many additional similar or wholly disparate tasks/purposes.

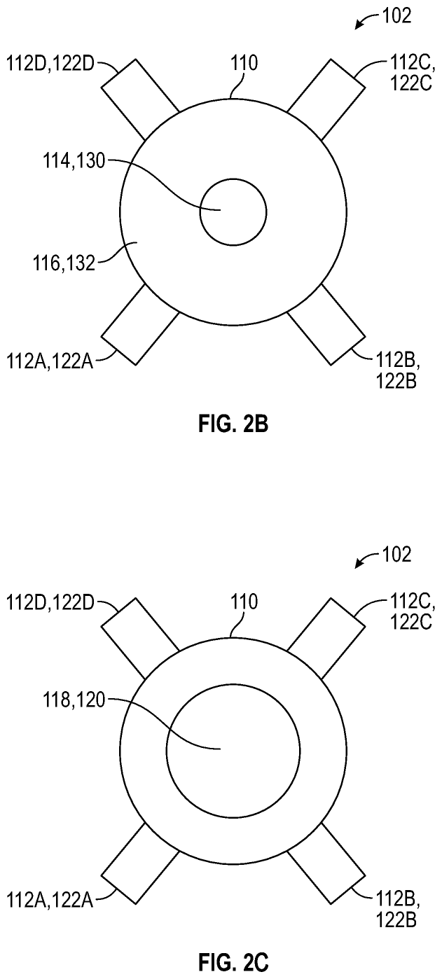

FIGS. 2A-2C show the smart implant 102 in further detail. In addition to the onboard electronics 104 including the one or more sensors 106 discussed previously and not specifically shown in FIGS. 2A-2C, the smart implant 102 can include a housing 110, one or more anchoring features 112A, 112B, 112C and 112D (shown only in FIGS. 2B and 2C) and one or more coupling features 114 (FIG. 2B).

The onboard electronics 104 including the one or more sensors 106 shown in FIG. 1 can be housed within and carried by the housing 110. The housing 110 can be formed of suitable material (e.g., metal, metal alloy, plastic, etc.) for implantation in the human body. The housing 110 can be generally cylindrical in shape having a proximal end portion 116 and a distal end portion 118. As shown in FIGS. 2A and 2C, the distal end portion 118 can include a taper 120 (such as in the form of a truncated cone) or other feature to facilitate insertion of the smart implant 102 into the medullary canal of the bone. The distal end portion 118 can also include a blunt tip at the end thereof. A diameter of the housing 110 can be between 10 mm and 75 mm, inclusive. The diameter can vary depending upon the relevant bone for which the smart implant 102 is designed to be inserted.

The housing 110 and the one or more anchoring features 112A, 112B, 112C and 112D can be sized and shaped as appropriate for insertion into and fixation within the medullary canal of the relevant bone (e.g., the tibia, femur, humerus, etc.). Thus, it is contemplated that the housing 110 and the one or more anchoring features 112A, 112B, 112C and 112D can be available as a system with different sizes and/or shapes according to some examples. The shape of the housing 110 and the one or more anchoring features 112A, 112B, 112C and 112D can be determined based on average medullary canal anatomy derived from three- or two-dimensional scans of the relevant bone using X-Ray, MRI, CT, ultrasound or other imaging techniques. Such shaping can include use of a large number of scans and the ZiBRA™ Anatomical Modeling System to analyze thousands of bones, both male and female, representing a diverse global population, for example. Alternatively, the shape of the housing 110 and the one or more anchoring features 112A, 112B, 112C and 112D can be patient-specific (i.e. is constructed specifically for the patient).

The one or more anchoring features 112A, 112B, 112C and 112D can project outward of the housing 110 and can be configured to engage the surface(s) of the bone that defines the medullary canal at various locations. Although four anchoring features 112A, 112B, 112C and 112D are illustrated, other examples contemplate the use of smaller number (one, two, three) or larger number (five or more) of features. Similarly, the relative positioning of the anchoring features 112A, 112B, 112C and 112D with respect to one another is purely exemplary.

The one or more anchoring features 112A, 112B, 112C and 112D can be any known mechanical feature (e.g., corrugations, porous elements, projections, fins, threads, tangs, prongs, tabs, hooks, loops, arms, apertures (e.g., slot, hole, etc.) or other known mechanical coupling feature) configured for facilitating anchoring to bone. Specifically, the one or more anchoring features 112A, 112B, 112C and 112D can be any known mechanical feature configured to anchor with the bone that forms medullary canal. The one or more anchoring features 112A, 112B, 112C and 112D are fins 122A, 122B, 122C and 122D in the example of FIGS. 1-2C. Additional of the figures provide other configurations for the anchoring features. The fins 122A, 122B, 122C and 122D can be configured to retain the device such that the smart implant 102 does not rotate or otherwise move within the medullary canal. It is important for the smart implant 102 to be immobilized to maintain a spatial relationship with other components during surgery such as cut guides, other tools, and/or implants.

The fins 122A, 122B, 122C and 122D can be rigid having a predefined shape and a fixed orientation that does not change substantially relative to the housing 110 or other components or anatomy. Alternatively, the fins 122A, 122B, 122C and 122D can be moveable (e.g., inward toward the housing 110 or outward away from the housing 110) as desired. Furthermore, the fins 122A, 122B, 122C and 122D can be configured to flex/deform against and conform with the surface of the bone, for example. Thus, the fins 122A, 122B, 122C and 122D can be formed of a shape memory or other flexible/conforming material if desired.

As shown in FIG. 2A, one or more of the fins 122A, 122B, 122C and 122D can include a first chamfered portion 124, a middle portion 126 and a second chamfered portion 128. The middle portion 126 can have the furthest extent outward of the housing 110. This location can provide a region of greatest thickness (radially outward extent from the housing 110) for the fins 122A, 122B, 122C and 122D. Such region of greatest thickness can extend outward of the housing by between 0.1 mm and 30 mm, inclusive. The first chamfered portion 124 and/or the second chamfered portion 128 can facilitate non-damaging engagement of the one or more of the fins 122A, 122B, 122C and 122D with the bone that forms the medullary canal. Put another way, the first chamfered portion 124 and/or the second chamfered portion 128 are configured as ramps to reduce the likelihood of the bone cracking upon insertion of the smart implant 102 into the medullary canal (i.e., the second chamfered portion 128 being in the insertion direction, i.e., it is on the leading end of the fins 122), and/or upon removal of the smart implant 102 (i.e., the first chamfered portion 124 being in the removal or withdrawal direction, i.e., it is on the trailing end of the fins 122).

As shown in FIG. 2A, the housing 110 can have an elongate length along axis L. Similarly, the fins 122A, 122B, 122C and 122D can have an elongate length as measured along the axis L as compared with the thickness and width thereof. This elongate length of each of the fins 122A, 122B, 122C and 122D can be on the order of 3× to 15× the thickness and/or width of each of the fins 122A, 122B, 122C and 122D. The fins 122A, 122B, 122C and 122D can be spaced equidistant around a circumference of the housing 110. The fins 122A, 122B, 122C and 122D can be located at the proximal end portion 116 of the housing 110. However, other examples contemplate the fins 122A, 122B, 122C and 122D being placed in other locations of the housing 110.

FIG. 2B is a plan view of the proximal end portion 116 of the housing 110 having the one or more coupling features 114. The one or more coupling features 114 can be a threaded aperture 130 extending into a proximal surface 132 of the housing 110 for example. The one or more coupling features 114 can alternatively be any known mechanical feature (e.g., male thread, projection, fin, thread, tang, prong, tab, hook, loop, arm, slot, etc.) configured for coupling with another component such as an insertion tool (e.g., screwdriver) and/or other tool (e.g., cut guide, threaded stud, etc.).

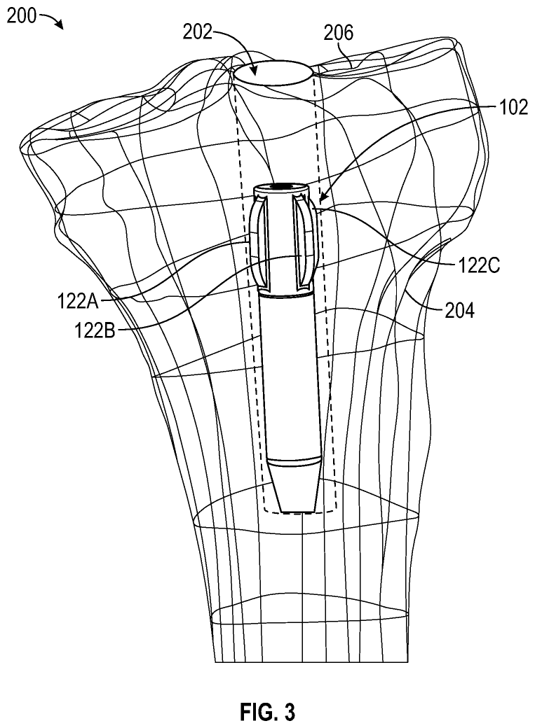

As shown in FIG. 3, the fins 122A, 122B and 122C (122D not visible) can be configured to engage with a tibia 204 along a medullary canal 202 thereof. According to one method 200, the medullary canal 202 can be reamed or otherwise formed in the tibia 204 as known in the art and the smart implant 102 can be inserted down into the medullary canal 202 to the location shown. The engagement of the fins 122A, 122B, 122C and 122D (the one or more anchoring features) can retain the smart implant 102 at a desired location along the medullary canal 202 a desired distance distal of an unresected proximal surface 206 of the tibia 204. This distance for the smart implant 102 distal of the unresected proximal surface 206 can be sufficient to allow for one or more resections of a proximal portion of the tibia 204 to remove the unresected proximal surface 206 as further described in co-pending cases entitled SYSTEMS, METHODS, AND APPARATUSES FOR TIBIAL MECHANICAL AXIS DIGITIZATION and COMPUTER-ASSISTED TIBIA RESECTION previously incorporated herein by reference. Stated differently, the smart implant 102 is deep enough in the medullary canal to have its trailing end below the resected tibial plateau.

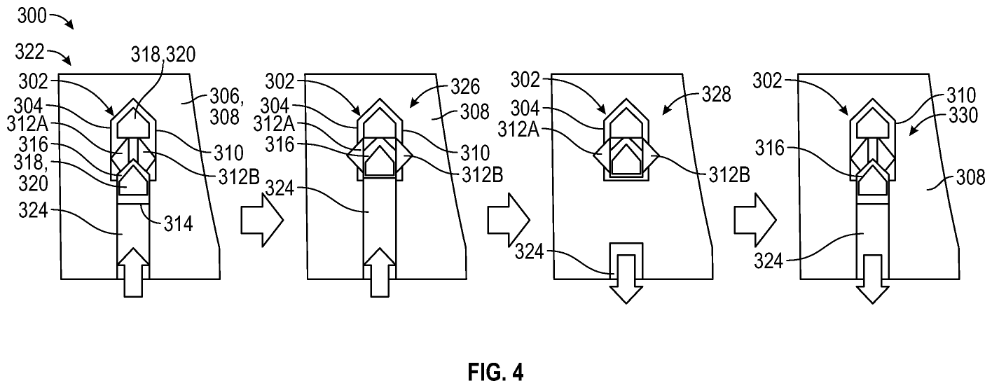

FIG. 4 shows a method 300 of anchoring a second example of a smart implant 302 temporarily within a medullary canal 304 of the long bone 306 such as a femur 308 or tibia. The smart implant 302 can include a housing 310, one or more anchoring features 312A and 312B, a coupling feature 314, an actuator 316 and onboard electronics 318 including one or more sensors 320.

Various of the components such as the housing 310, the onboard electronics 318 including the one or more sensors 320 can be the same or similar in construction to any of those described herein. The onboard electronics 318 including the one or more sensors 320 can be housed within the housing 310 and/or the actuator 316, for example. The actuator 316 can have the coupling feature 314 with a construction as described previously. The method 300 can include inserting at step 322 the smart implant 302 into the medullary canal 304 such as with an inserter 324 (e.g., screwdriver, etc.) coupled to the actuator 316 via the coupling feature 314. Initially, at step 322, upon insertion, the smart implant 302 can have a first configuration. In this first configuration, the actuator 316 can project distally from the housing 310 and the one or more anchoring features 312A and 312B (e.g., wings, fins, wedges, etc.) can be housed internally within the housing 310. At step 326, the actuator 316 has been moved proximally by the inserter 324 into the housing 310 to a second position and the smart implant 302 has a second configuration. This movement and engagement of the actuator 316 causes the one or more anchoring features 312A and 312B to be extended outward of the housing 310 and to engage the femur 308 along the medullary canal 304. The anchoring features 312A and 312B may thus be deployed in retracted. This applies as well to the fins 122A to 122D in the embodiment of FIGS. 2A to 2C. At step 328, the inserter 324 can be decoupled from the smart implant 302 and can be removed from the femur 308. The smart implant 302 is anchored against rotation and other movement in the medullary canal 304 by the one or more anchoring features 312A and 312B. The method 300 contemplates sensing one or more patient characteristics including but not limited to orientation of the smart implant 302 as dictated by the orientation of the bone, movement of the smart implant 302 as dictated by movement of the bone, temperature, pH, etc. using the one or more sensors 320. After sensing, resection of the femur 308 and/or other trialing or surgical steps are performed, the method 300 at step 330 recouples the inserter 324 to the actuator 316 and withdraws at least a portion of the actuator 316 distal of the housing 310. This action, along with pressure within the medullary canal, can retract the one or more anchoring features 312A and 312B partially or fully back into the housing 310 such that the smart implant 302 can disengage from the femur 308 and can be removed from the medullary canal 304 by the inserter 324 without the one or more anchoring features 312A and 312B contacting the femur 308. The actuator 316 may be a passive actuator, actuated manually from a tool such as the inserter 324.

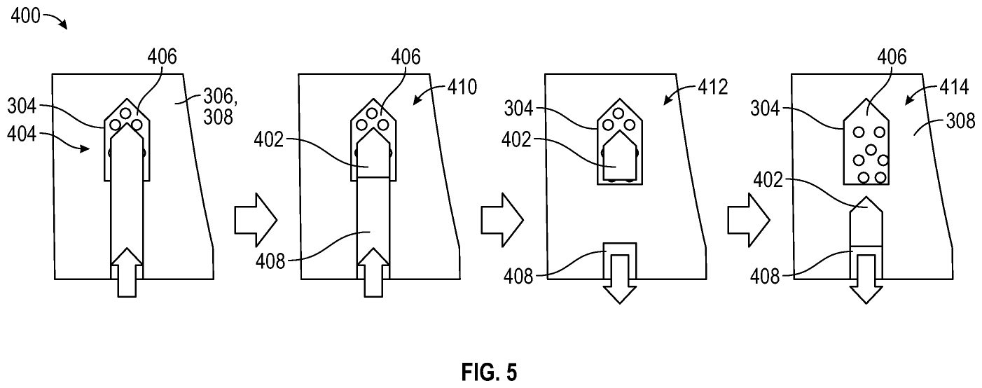

FIG. 5 shows a method 400 of anchoring a third example of a smart implant 402 temporarily within the medullary canal 304 of the long bone 306 such as the femur 308 or tibia. For simplicity, the smart implant 402 does not specifically illustrate or reference components such as the housing, one or more sensors, etc. It is understood that the smart implant 402 can have any of the components discussed herein.

The method 400 includes at step 404 a porous shell 406 that is mounted onto an inserter 408 and inserted into the medullary canal 304. The porous shell 406 can be filled with bone cement. After insertion at step 404, the bone cement can be allowed to harden to fixate the porous shell 406 within the medullary canal 304. At step 410, the method 400 can include mounting the smart implant 402 onto the inserter 408 and anchoring the smart implant 402 within the porous shell 406. At step 412, the inserter 408 can be removed and the smart implant 402 can be left behind within the medullary canal 304 for sensing one or more patient characteristics including but not limited to orientation of the smart implant 502 as dictated by orientation of the bone, movement of the smart implant 302 as dictated by movement of the bone, temperature, pH, etc. during the surgery. After sensing, resection of the femur 308 and/or other trialing or surgical steps are performed, the method 400 at step 414 recouples the inserter 408 to the smart implant 402 and withdraws the smart implant 402 from the femur 308 leaving the porous shell 406 behind in the medullary canal 304.

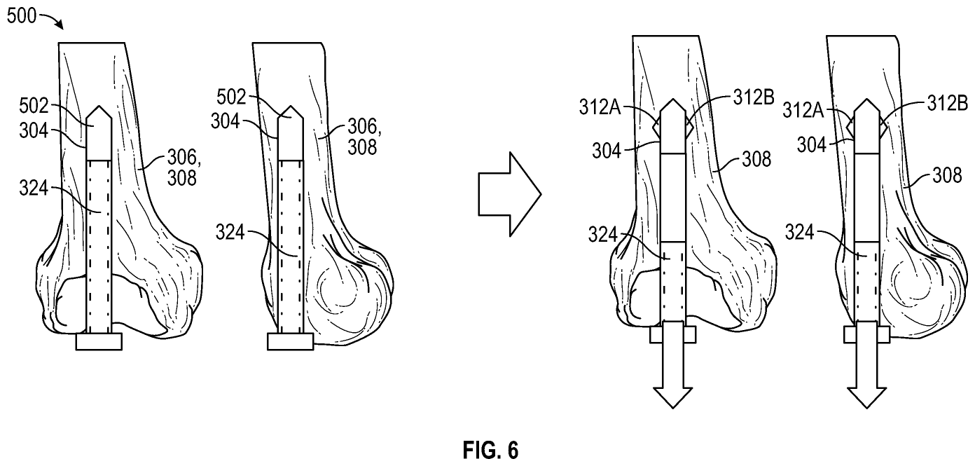

FIG. 6 shows a method 500 of anchoring a fourth example of a smart implant 502 temporarily within the medullary canal 304 of the long bone 306 such as the femur 308 or tibia. For simplicity, the smart implant 502 does not specifically illustrate or reference components such as the housing, one or more sensors, etc. It is understood that the smart implant 502 can have any of the components discussed herein.

The method 500 can be similar to the method 300 as previously described and utilizes the one or more anchoring features 312A and 312 to be extended outward of the housing 310 and to engage the femur 308 along the medullary canal 304. This extending of the one or more anchoring features 312A and 312B can be facilitated by rotation or other actuation by the inserter 324. The method 500 can include sensing one or more patient characteristics including but not limited to orientation of the smart implant 502 as dictated by orientation of the bone, movement of the smart implant 502 as dictated by movement of the bone, temperature, pH, etc. during the surgery. The method 500 differs from the method 300 in that the inserter 324 can be retained at least partially in the femur 308 (but decoupled from the smart implant 502) during portions or all of the surgery. Indeed the inserter 324 can remain coupled to the smart implant 502 in some cases during all or part of the trialing. The inserter 324 can provide a retention feature for other components such as a cut guide in the manner of an intramedullary rod, for example.

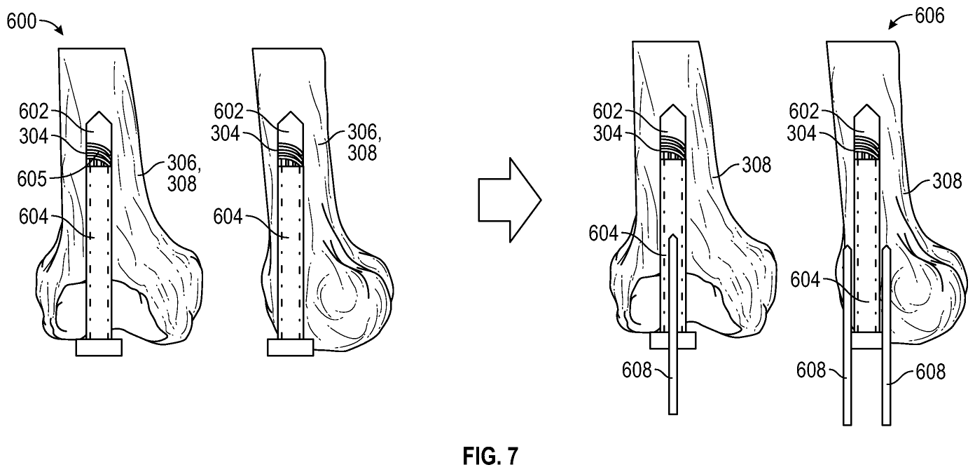

FIG. 7 shows a method 600 of anchoring a fifth example of a smart implant 602 temporarily within the medullary canal 304 of the long bone 306 such as the femur 308. For simplicity, the smart implant 602 does not specifically illustrate or reference components such as the housing, one or more sensors, etc. It is understood that the smart implant 602 can have any of the components discussed herein.

As shown in FIG. 7 anchoring of the smart implant 602 within the medullary canal 304 is performed by an inserter 604, which remains engaged with the smart implant 602 during all or part of the surgery. Thus, the inserter 604 acts as the one or more anchoring features according to the method of FIG. 7. As shown in FIG. 7 coupling of the smart implant 602 and the inserter 604 can be via threading 605 of other mechanical coupling features. The threading 605 can also be used to anchor the smart implant 602 within the medullary canal.

The method 600 can include sensing one or more patient characteristics including but not limited to orientation of the smart implant 602 as dictated by movement of the bone, movement of the smart implant 602 as dictated by movement of the bone, temperature, pH, etc. during the surgery. The inserter 604 can extend from the smart implant 602 to an external location outside of the femur 308. Optionally at step 606 one or more pins 608 can be added adjacent the inserter 604. The inserter 604 and/or the pins 608 can provide a retention feature for other components such as a cut guide. The pins 608 may or may not be connected to the inserter 604.

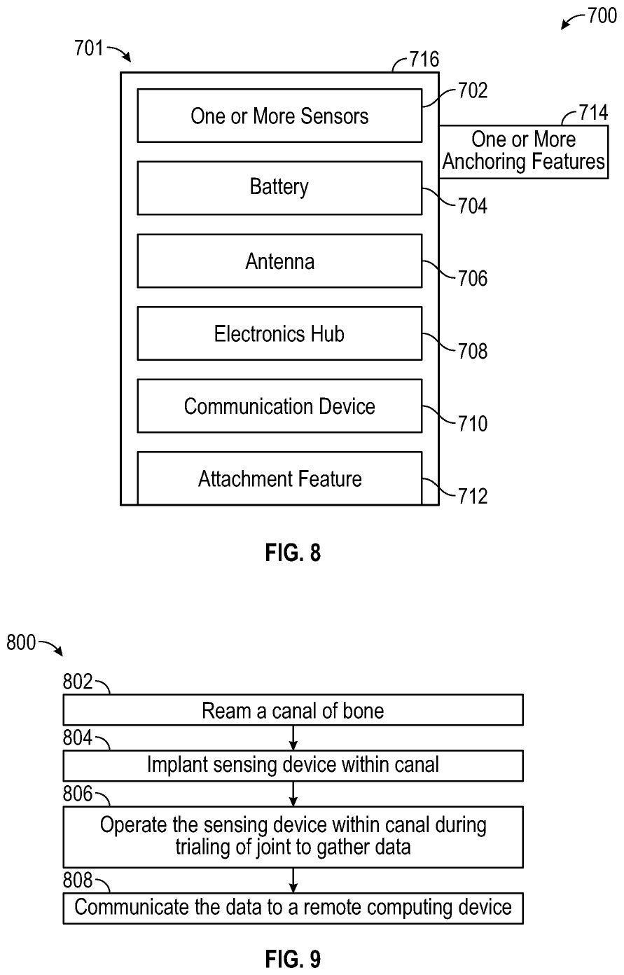

FIG. 8 shows a highly schematic view of a smart implant 700 with various mechanical and electronic components. These components may be in any of the smart implants described herein. The smart implant 700 can include onboard electronics 701 including one or more sensors 702, a battery 704, an antenna 706, an electronics hub 708, and a communication device 710. The mechanical components can include an attachment feature 712 (also called a coupling feature or coupling mechanism herein), one or more anchoring features 714 and a housing 716.

The smart implant 700 can comprise a self-contained sensing unit configured to put the one or more sensors 702 in contact with or in proximity to various anatomic features such as the medullary canal, knee joint, etc. as previously discussed and illustrated. The one or more anchoring features 714 are configured to facilitate engagement and coupling to the bone within the medullary canal as previously described. For example, different instances of the one or more anchoring features 714 can have different exterior form factor for mating with different anatomies, such as different long bones or other bones of the patient.

The housing 716 can comprise a body in which components of smart implant 700 including the one or more sensors 702, the battery 704, the antenna 706, the electronics hub 708, and the communication device 710 can be located. In examples, the housing 716 can be sealed to prevent liquid or biological matter from entering the interior of the housing 716. However, other examples including some that are discussed herein contemplate the housing having apertures or other features and others of the one or more sensors 702, the battery 704, the antenna 706, the electronics hub 708, and the communication device 710 being sealed. The housing 716 can include one or more ports that can comprise interfaces with surrounding tissue, biological matter or a medical device. These port(s) can allow fluid to penetrate the housing 716 to engage a component of the one or more sensors 702. Port(s) can comprise an electrode or interface component of one or more sensors 702 that is extended into port to engage with the surrounding environment of housing 716. In examples, housing 716 can have a diameter in the range of approximately 10-12 mm and can have a length in the range of approximately 30-40 mm.

The one or more anchoring features 714 can comprise a feature of or a portion of the housing 716 configured to facilitate engagement with tissue such as the bone of the medullary canal. Alternatively, the one or more anchoring features 714 can comprise a component that is separate from the housing 716. Examples of separate anchoring features from the housing are provided in FIGS. 4 and 6 where the anchoring features are extendible and retractable relative to the housing.

The one or more anchoring features 714 can comprise any type of mechanical feature/mechanism configured to facilitate coupling of the smart implant 700 to the anatomy. As discussed previously, the one or more anchoring features 714 can transform, move or can be shaped such that the outer perimeter shape or form factor of smart implant 700 can better mate with anatomy into which smart implant 700 is inserted into. The one or more anchoring features 714 can facilitate engagement with tissue, such as cortical bone. The one or more anchoring features 714 can facilitate engagement with medullary canals of long bones and other tissue. The one or more anchoring features 714 and/or the housing 716 can have other shapes to fit with other anatomical features, such as a spherical shape, a cylindrical shape, a disk shape, a cup shape and others to mate with other anatomic features of different sized intramedullary canals. As such, a surgeon can select the type of smart implant 700 to use with specific anatomic features or patients.

The housing 716 can comprise an easily manufactured shape such as a cylindrical or capsule shape. However, the housing 716 can have a customized or irregularly shaped geometry to mate with particular anatomic features.

The one or more sensors 702 can be any one or combination of different types of sensors (e.g., an accelerometer, a gyroscope, a piezoelectric sensor, force sensor, thermometer, pH monitor, strain gauge, or any combination thereof, including any other sensor that can be used to detect motion within a body). As such, it is contemplated one or more sensors 702 can be two or more sensors with coordinated orientation data that can be communicated outside of the patient. Coordinated data regarding the motion of the bone, orientation of the bone can be used to evaluate the range of motion of the joint. Furthermore, the one or more sensors 702 can include a motion sensor, such as a 3-axis accelerometer or magnetic Hall effect sensors and the like.

The electronics hub 708 can include a circuit board for electrically and structurally coupling electronic components of the smart implant 700. For example, electronics hub 708 can comprise a silicon wafer or a chip onto which electrical couplings are attached for coupling with other components (e.g., a switch, processor, memory, the one or more sensors 702 and the like. The processor can comprise an integrated circuit that controls operation of components of smart implant 700, such as I/O device, the communication device 710 and the one or more sensors 702, etc. The processor can execute instructions stored in memory to operate components of smart implant 700, such as the one or more sensors 702.

The memory can comprise any suitable storage device, such as non-volatile memory, magnetic memory, flash memory, volatile memory, programmable read-only memory and the like. The memory can include instructions stored therein for the processor to control operation of smart implant 700. For example, memory can include instructions for operating I/O device, communication device 710 and the one or more sensors 702, as well as coordinating output from smart implant 700. Memory can additionally include reference data for comparing data from the one or more sensors 702.

The communication device 710 can comprise one or more devices for receiving input from an interrogation device (e.g., the controller 108 of FIG. 1) or providing an output to interrogation device via various signals. The communication device 710 can provide a signal to the interrogation device. The interrogation device can thereafter, for example, display on human interface device, such as a video display monitor, an indication of information from the smart implant 700.

The communication device 710 can receive a signal from the interrogation device for storing information on memory or providing information to processor for operating the one or more sensors 702 and other electronics components. In examples, the communication device 710 can communicate using wireless communications signals, such as Bluetooth, WiFi, Zigbee, infrared (IR), near field communication (NFC), 3GPP or other technologies. In examples, the communication device can comprise a wired connection or can include a port for receiving a wire for a wired connection.

The communication device 710 can be used in conjunction with the antenna 706. The battery 704 can comprise a power source. The battery 704 can include an electrochemical cell, such as an alkaline or zinc-manganese battery. In examples, power source can comprise a primary, or non-rechargeable battery, a rechargeable battery or another type of power source.

FIG. 9 is a flow diagram of a method 800 of monitoring one or more characteristics of a patient using a sensing device such as smart implant of any of the examples provided herein. The method 800 can be utilized only during trialing of the joint, for example. Thus, the sensing device can be a temporary implant that is removed upon conclusion of trialing and before permanent implants are mounted to replicate the joint.

The method 800 can include reaming 802 a medullary canal of a bone, or providing access to the medullary canal in any appropriate way. The method 800 can include implanting 804 the sensing device within the medullary canal of the bone. The method 800 can include operating 806 the sensing device during the trialing of the joint of the patient while sensing device is implanted within the medullary canal to generate data regarding the one or more characteristics of the patient (e.g., orientation of the bone, movement of the bone, temperature within the medullary canal, pH within the medullary canal, and/or other data). The method 800 can include 808 communicating the generated data from the sensing device to a remote computing device external of the patient (e.g., the controller 108 of FIG. 1). The method 800 can optionally include that the implanting the sensing device within the medullary canal of the bone includes anchoring the sensing device within the medullary canal using at least one of one or more anchoring features (examples discussed and illustrated previously). The one or more anchoring features can comprise a plurality of fins spaced around a circumference of the sensing device. Additionally, or alternatively, the implanting the sensing device within the medullary canal of the bone can include extending one or more anchoring features to abut against the bone within the medullary canal to anchor the sensing device within the medullary canal. Additionally, or alternatively, the implanting the sensing device within the medullary canal of the bone can include anchoring the sensing device within the medullary canal using a tool coupled to the sensing device. The anchoring the sensing device within the medullary canal using the tool can include actuating with the tool a component of the sensing device to extend one or more anchoring features against the bone within the medullary canal. The method 800 can further include removing the sensing device from the medullary canal after the trialing of the joint is complete and prior to resection of the bone of the patient. The removing the sensing device from the medullary canal after the trialing of the joint is complete can further include retracting one or more anchoring features from contact with the bone within the medullary canal.

The systems, devices and methods discussed in the present application can be useful in efficiently and inexpensively implanting sensing capabilities into a patient in conjunction with a CAS system. As discussed herein, the smart implant can be adapted for use with different anatomies. The smart implant can be temporarily implanted in a patient during trialing such as at the medullary canal of the bone to sense one or more characteristics such as orientation of the bone, movement of the bone, temperature within the medullary canal, pH within the medullary canal, and/or other data.

### Claim Related Examples

To further illustrate the apparatuses, systems and methods disclosed herein, the following non-limiting examples (referred to below as aspects and/or techniques) are provided:

In some aspects, the techniques described herein relate to an implantable device for collecting data regarding one or more characteristics of a bone of a patient, the implantable device can optionally include: a housing; onboard electronics including one or more sensors carried by the housing; and one or more anchoring features coupled to the housing and extending outward thereof, wherein the one or more anchoring features are configured to engage with the bone at a medullary canal thereof to couple the implantable device with the bone. The one or more anchoring features are moveable from a first position, where the one or more anchoring features are positioned in the housing, to a second position, where the one or more anchoring features extend outward of the housing.

In some aspects, the techniques described herein relate to an implantable device, wherein the one or more anchoring features include a plurality of fins spaced around a circumference of the housing.

In some aspects, the techniques described herein relate to an implantable device, wherein the one or more anchoring features include at least a first chamfered portion.

In some aspects, the techniques described herein relate to an implantable device, wherein the one or more anchoring features are moveable from a first position, where the one or more anchoring features positioned in the housing, to a second position, where the one or more anchoring features extend outward of the housing.

In some aspects, the techniques described herein relate to an implantable device, further including an actuator configured to extend and retract the one or more anchoring features.

In some aspects, the techniques described herein relate to an implantable device, further including a coupling feature configured to couple the implantable device to a tool.

In some aspects, the techniques described herein relate to an implantable device, wherein the one or more anchoring features include a tool that extends to external of the medullary canal of the bone.

In some aspects, the techniques described herein relate to a system configured to be implanted into a patient, the system can optionally include: an implantable device having one or more sensors device for collecting data regarding one or more characteristics of a bone of a patient and one or more anchoring features configured to engage with the bone at a medullary canal thereof to couple the implantable device with the bone in the medullary canal; and a tool configured to couple with the implantable device within the medullary canal.

In some aspects, the techniques described herein relate to a system, wherein the one or more anchoring features include a plurality of fins spaced around a circumference of the implantable device.

In some aspects, the techniques described herein relate to a system, wherein the one or more anchoring features include at least a first chamfered portion.

In some aspects, the techniques described herein relate to a system, wherein the one or more anchoring features are moveable by the tool from a first position, where the one or more anchoring features positioned in a housing of the implantable device, to a second position, where the one or more anchoring features extend outward of the housing.

In some aspects, the techniques described herein relate to a system, wherein the tool is configured to anchor the implantable device within the medullary canal.

In some aspects, the techniques described herein relate to a method of monitoring one or more characteristics of a patient, the method can optionally include: reaming a medullary canal of a bone; implanting a sensing device within the medullary canal of the bone; operating the sensing device during a trialing of a joint of the patient while sensing device is implanted within the medullary canal to generate data regarding the one or more characteristics of the patient; and communicating the generated data from the sensing device to a remote computing device external of the patient.

In some aspects, the techniques described herein relate to a method, wherein implanting the sensing device within the medullary canal of the bone includes anchoring the sensing device within the medullary canal using at least one of one or more anchoring features.

In some aspects, the techniques described herein relate to a method, wherein the one or more anchoring features include a plurality of fins spaced around a circumference of the sensing device.

In some aspects, the techniques described herein relate to a method, wherein implanting the sensing device within the medullary canal of the bone includes extending one or more anchoring features to abut against the bone within the medullary canal to anchor the sensing device within the medullary canal.

In some aspects, the techniques described herein relate to a method, wherein implanting the sensing device within the medullary canal of the bone includes anchoring the sensing device within the medullary canal using a tool coupled to the sensing device.

In some aspects, the techniques described herein relate to a method, wherein anchoring the sensing device within the medullary canal using the tool includes actuating with the tool a component of the sensing device to extend one or more anchoring features against the bone within the medullary canal.

In some aspects, the techniques described herein relate to a method, further including removing the sensing device from the medullary canal after the trialing of the joint is complete and prior to resection of the bone of the patient.

In some aspects, the techniques described herein relate to a method, wherein the removing the sensing device from the medullary canal after the trialing of the joint is complete further includes retracting one or more anchoring features from contact with the bone within the medullary canal.

Each of these non-limiting examples can stand on its own, or can be combined in various permutations or combinations with one or more of the other examples. These and other examples and features of the present apparatuses, systems and methods will be set forth in part in the Detailed Description.

### Various Notes

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventor also contemplates examples in which only those elements shown or described are provided. Moreover, the present inventor also contemplates examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1. An implantable device for collecting data regarding one or more characteristics of a bone of a patient, the implantable device comprising:
a housing;
onboard electronics including one or more sensors carried by the housing; and
one or more anchoring features coupled to the housing and extending outward thereof,
wherein the one or more anchoring features are configured to engage with the bone at a medullary canal thereof to couple the implantable device with the bone; and
wherein the one or more anchoring features are moveable from a first position, where the one or more anchoring features are positioned in the housing, to a second position, where the one or more anchoring features extend outward of the housing.

2. The implantable device of claim 1, wherein the one or more anchoring features comprise a plurality of fins spaced around a circumference of the housing.

3. The implantable device of claim 2, wherein the one or more anchoring features include at least a first chamfered portion in an insertion direction.

4. The implantable device of claim 3, wherein the one or more anchoring features include at least a second chamfered portion in a removal direction.

5. The implantable device of claim 1, further comprising an actuator configured to extend and retract the one or more anchoring features.

6. The implantable device of claim 1, further comprising a coupling feature configured to couple the implantable device to a tool.

7. The implantable device of claim 1, wherein the one or more anchoring features comprise a tool that extends to external of the medullary canal of the bone.

8. A system configured to be implanted into a patient, the system comprising:
an implantable device having one or more sensors device for collecting data regarding one or more characteristics of a bone of a patient and one or more anchoring features configured to engage with the bone at a medullary canal thereof to couple the implantable device with the bone in the medullary canal; and
a tool configured to releasably couple with the implantable device within the medullary canal.

9. The system of claim 8, wherein the one or more anchoring features comprise a plurality of fins spaced around a circumference of the implantable device.

10. The system of claim 9, wherein the one or more anchoring features include at least a first chamfered portion.

11. The system of claim 8, wherein the one or more anchoring features are moveable by the tool from a first position, where the one or more anchoring features positioned in a housing of the implantable device, to a second position, where the one or more anchoring features extend outward of the housing.

12. The system of claim 8, wherein the tool is configured to anchor the implantable device within the medullary canal.

13. A method of monitoring one or more characteristics of a patient, the method comprising:
reaming a medullary canal of a bone;
implanting a sensing device within the medullary canal of the bone;
operating the sensing device during a trialing of a joint of the patient while sensing device is implanted within the medullary canal to generate data regarding the one or more characteristics of the patient; and
communicating the generated data from the sensing device to a remote computing device external of the patient.

14. The method of claim 13, wherein implanting the sensing device within the medullary canal of the bone includes anchoring the sensing device within the medullary canal using at least one of one or more anchoring features.

15. The method of claim 14, wherein the one or more anchoring features comprise a plurality of fins spaced around a circumference of the sensing device.

16. The method of claim 13, wherein implanting the sensing device within the medullary canal of the bone includes extending one or more anchoring features to abut against the bone within the medullary canal to anchor the sensing device within the medullary canal.

17. The method of claim 13, wherein implanting the sensing device within the medullary canal of the bone includes anchoring the sensing device within the medullary canal using a tool coupled to the sensing device.

18. The method of claim 17, wherein anchoring the sensing device within the medullary canal using the tool includes actuating with the tool a component of the sensing device to extend one or more anchoring features against the bone within the medullary canal.

19. The method of claim 13, further comprising removing the sensing device from the medullary canal after the trialing of the joint is complete and prior to resection of the bone of the patient.

20. The method of claim 19, wherein the removing the sensing device from the medullary canal after the trialing of the joint is complete further includes retracting one or more anchoring features from contact with the bone within the medullary canal.
